(19) United States
(12) Patent Application Publication
(10) Pub. No.: US 2011/0029086 A1
(43) Pub. Date: Feb. 3, 2011

(54) LUMBAR JACK IMPLANT

(76) Inventors: Paul A. Glazer, Chestnut Hill, MA (US); Lawrence Crainich, Charlestown, NH (US)

Correspondence Address: EDWARDS ANGELL PALMER & DODGE LLP, P.O. BOX 55874, BOSTON, MA, 02205, US

(21) Appl. No.: 12/846,413

(22) Filed: Jul. 29, 2010

Related U.S. Application Data
(60) Provisional application No. 61/229,528, filed on Jul. 29, 2009.

(51) Int. Cl. A61F 2/44

(52) U.S. Cl. 623/17.16

(57) ABSTRACT

A spinal implant has a first bone-engaging member, a second bone-engaging member, and a linkage coupling the first bone-engaging to the second bone-engaging member. The implant includes means for receiving a tensile force to extend the implant.

### CROSS-REFERENCE TO RELATED APPLICATIONS

This application claims the benefit of U.S. Provisional Application No. 61/229,528, filed Jul. 29, 2009, which is hereby incorporated by reference in its entirety herein.

### FIELD OF THE INVENTION

The invention relates to spinal implants. More particularly, the invention relates to spacer implants.

### BACKGROUND OF THE INVENTION

Deterioration of the lumbo-sacral joint can cause debilitating strain on the spinal cord. Disk degeneration may cause relative displacement of the fifth lumbar vertebra L-5 relative to the vertebra S-1 of the sacrum, imposing a shearing strain on the spinal cord. Surgical operations to address this may shift the L-5 vertebra relative back to its normal position and fix it in place. An exemplary operation involves disk removal and insertion of a spacer implant in place thereof. To facilitate this, the L-5 vertebra may be manually pried away from (and into a more proper alignment with) the S-1 vertebra to allow the disk removal, vertebral surfacing (if any), and implant insertion.

### SUMMARY OF THE INVENTION

One aspect of the invention involves a spinal implant having a first bone-engaging member, a second bone-engaging member, and a linkage coupling the first bone-engaging to the second bone-engaging member. The implant includes means for receiving a tensile force to extend the implant.

In various implementations, the linkage may be a four-bar linkage with the first and second bone-engaging members forming respective first and second bars. The means may comprise a hook on one of the first and second members and a socket on the other. The implant may further comprise an adjuster having a first portion engaging the hook and a second portion engaging the socket.

The implant may be installed by using a tensile adjustment tool engaging the means to extend the implant. The implant may be shimmed and the tool removed. A lock may be installed in place of the tool. The lock may be an adjuster which may be tightened to further extend the implant and the shim removed.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a view of an implant and installation/extension tool.

FIG. 2 is a partial side view of the implant and tool of FIG. 1.

FIG. 3 is a back view (from the point of view of a patient) of the implant and tool of FIG. 1.

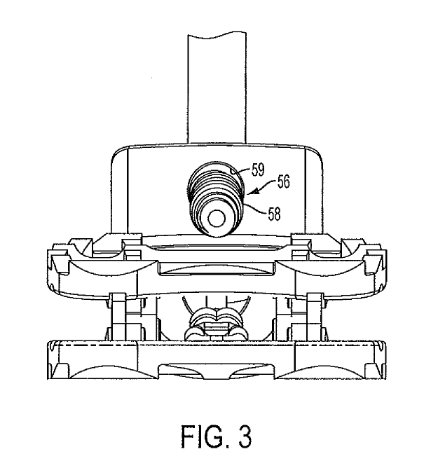

FIG. 4 is a top view of the tool and implant of FIG. 1.

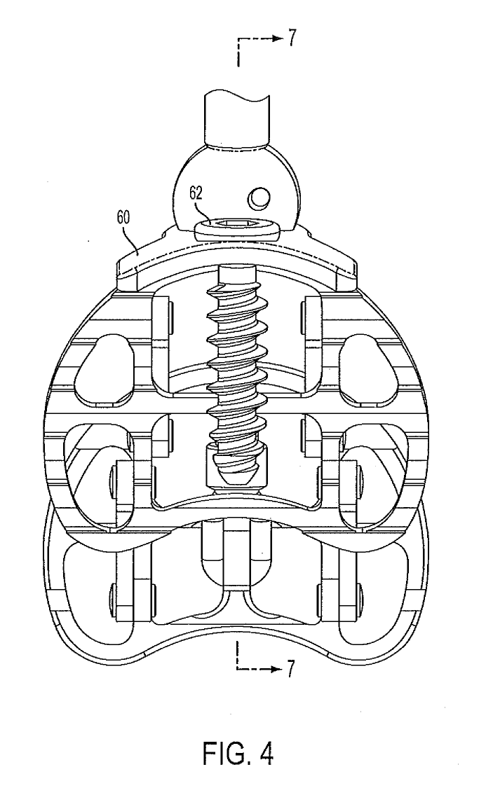

FIG. 5 is a bottom view of the tool and implant of FIG. 1.

FIG. 6 is a front view of the tool and implant of FIG. 1.

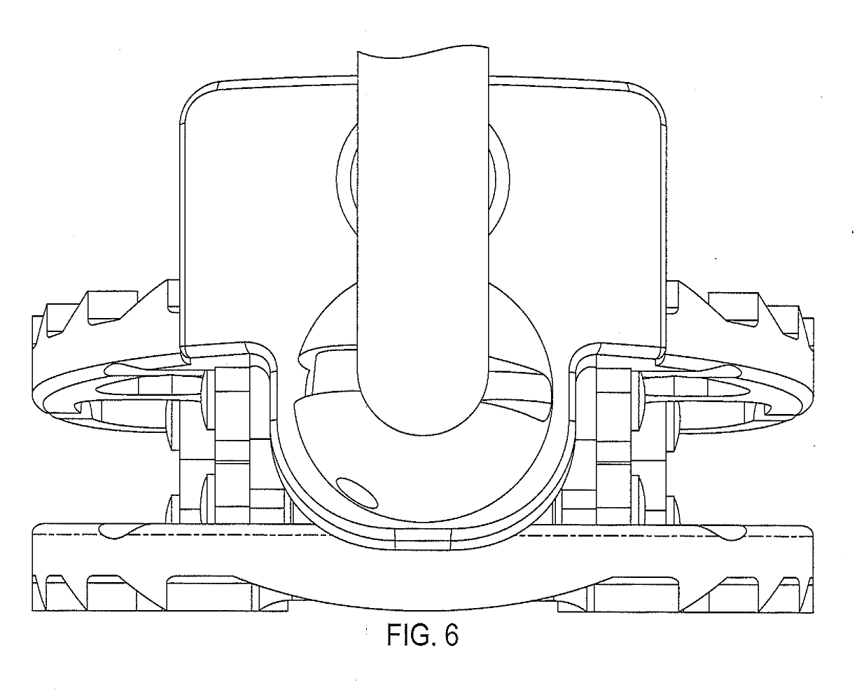

FIG. 7 is a longitudinal/vertical sectional view of the implant and tool taken along line 7-7 of FIG. 4.

FIG. 8 is a view of the implant of FIG. 1 with an adjuster installed in place of the tool.

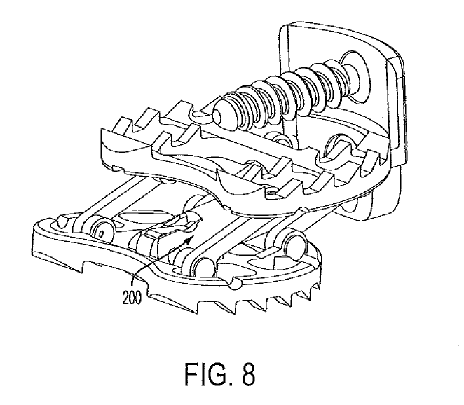

FIG. 9 is a top view of the implant of FIG. 8.

FIG. 10 is a longitudinal/vertical sectional view of the implant taken alone line 10-10 of FIG. 9.

FIG. 11 is a view of the implant in an extended condition.

FIG. 12 is a side view of the implant of FIG. 11.

Like reference numbers and designations in the various drawings indicate like elements.

### DETAILED DESCRIPTION

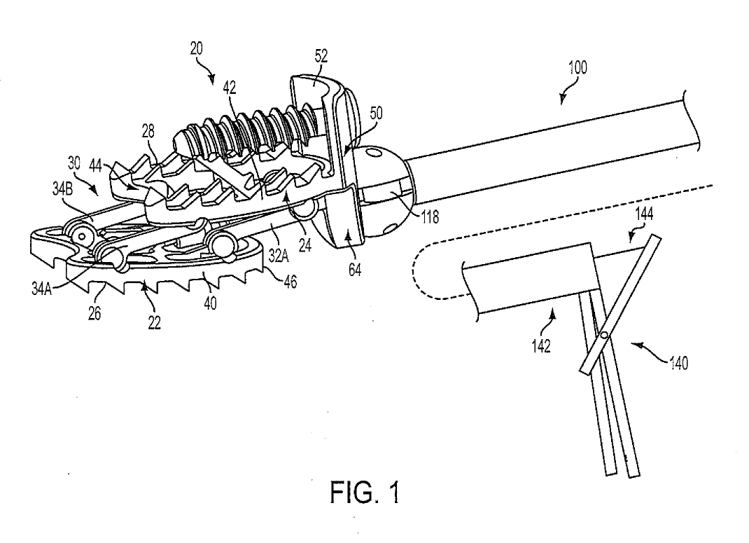

FIG. 1 shows an implant 20.

For ease of reference, a frame of reference identifying: upper and lower; left and right; and front and back (including equivalents) is taken relative to an installed condition with the patient standing upright, and the directions being the directions from the point of view of the patient (rather than someone standing in front of the patient and facing the patient).

The exemplary implant 20 has a lower bone-engaging member 22 (a bottom member or base) and an upper bone-engaging 24 (e.g., a top member). As is discussed further, the exemplary members may be articulated between a fully compressed/contracted/closed condition and a fully extended/expanded/opened condition. Insertion/installation may be in a relatively compressed condition (e.g., including the fully compressed condition) with subsequent expansion. The lower member has a bottom surface (underside) 26 for engaging an upper surface of the adjacent bone below (e.g., the sacrum). The upper member has a top surface 28 for engaging an underside of the adjacent bone above (e.g., the L-5 vertebra).

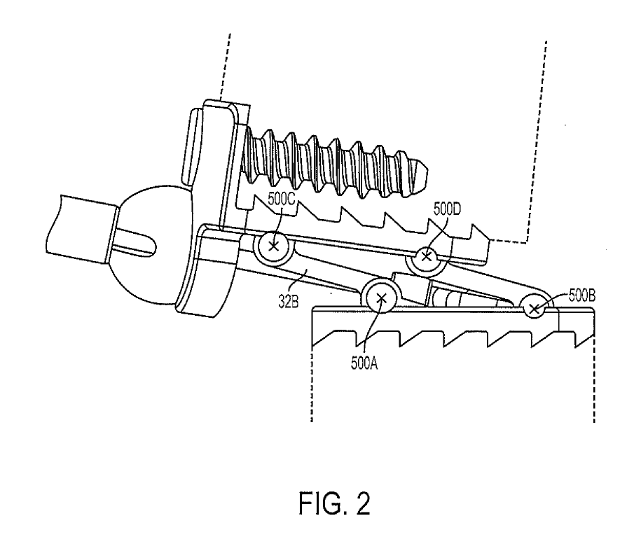

A linkage 30 guides the articulation between compressed and extended conditions. The exemplary linkage is a four-bar linkage wherein: the lower member forms a first bar; the upper member forms a second bar; one or more forward links 32A, 32B form a third bar; and one or more rear links 34A, 34B form the fourth bar. The third and fourth bars each have fixed pivots with associated pivot axes 500A, 500B, 500C, 500D (FIG. 2) relative to the first and second bars.

The exemplary lower and upper members each have lateral peripheral surfaces 40 and 42. The peripheral surfaces are characterized by a central rear concavity 44 defining a partial channel for accommodating the spinal cord. The members 22 and 24 have a planform generally corresponding to the adjacent bone and/or replaced disk. A forward portion 46 is thus convex.

The upper member, at its forward end, has an upwardly-extending flange or partial collar 50 having a concave inboard surface 52 for mating with the forward surface of the adjacent bone. The flange 50 has an aperture/hole 54 for receiving the shaft/shank 58 of a screw 56 to secure the upper member 24 to the bone above. The outboard surface 60 of the flange may have a counterbore for receiving the head 62 of the screw. The screw may have features for engaging a tool/driver (e.g., a hex socket feature).

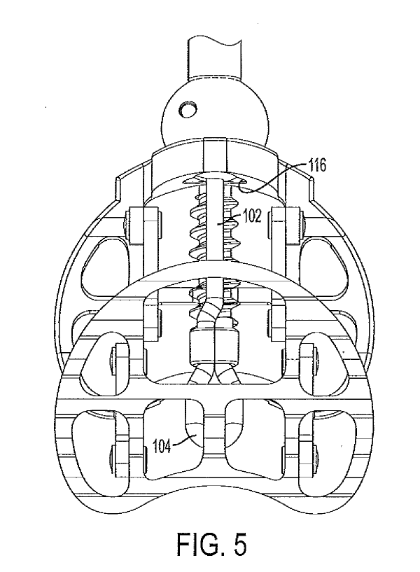

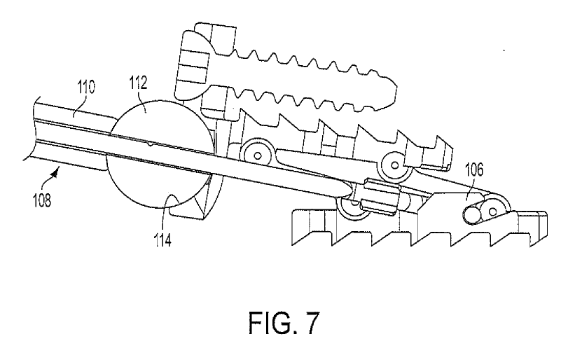

In an exemplary use, the patient's site is prepared (e.g., in a conventional manner). This preparation may include prying apart adjacent bones (e.g., sacrum and L-5 vertebra), disk removal, and preparation (e.g., resurfacing) of mating surfaces of the adjacent bones. The implant is installed in a relatively compressed condition (e.g., with the upper member racked forward relative to the lower member). The flange of the upper member may then be secured to the vertebra via the screw (e.g., into a pre-drilled hole in the vertebra). Alternatively, the hole may be drilled and screw installed after implant extension (described below). The implant may then be further extended. An exemplary extension is performed via an extension tool 100 (FIG. 1) which has been preinstalled to the implant (i.e., installed to the implant prior to placing the implant between the sacrum and vertebra). The exemplary tool operates by placing diagonal tension across the linkage. The exemplary tension is applied between the upper member 24 and lower member 22. The exemplary tension is applied via a cable 102 (FIG. 5) having a terminal loop 104 engaging a hook 106 (FIG. 7) on the lower member near a rear end thereof. The counterforce is applied by a guide 108 through which the cable passes. The exemplary guide comprises a rigid tube 110 extending to an end portion formed as a ball 112 abutting the upper member. The ball is received in a socket 114 of the upper member (e.g., in a lower flange/collar 64 below the collar 50) to allow relative rotation. The cable passes through a hole 116 (FIG. 5) at the base of the socket. The ball may carry one or more rollers 118 to engage the cable during a lateral rotation of the tool relative to the implant.

An actuator 140 (FIG. 1) may be formed at the proximal end 142 of the tool to draw the cable through the guide and, thereby extend the implant. The exemplary actuator is an inverted scissor action handgrip engaging a second end 144 of the cable. Alternative actuators may include rotary grips, ratcheting handgrips, and the like.

Tightening of the cable draws the lower member forward, with the racking action of the four-bar linkage then driving it downward. During this movement, the interaction of the teeth on the underside of the lower member cause the adjacent bone to be drawn along with the lower member to restore anatomical alignment of the bones. When a sufficient degree of extension has been reached, one or more shims may be inserted between upper and lower members, allowing the cable tension to be released with the shim supporting the members in a relatively extended condition. Further extension of the cable will release it from the hook allowing the tool to be disengaged.

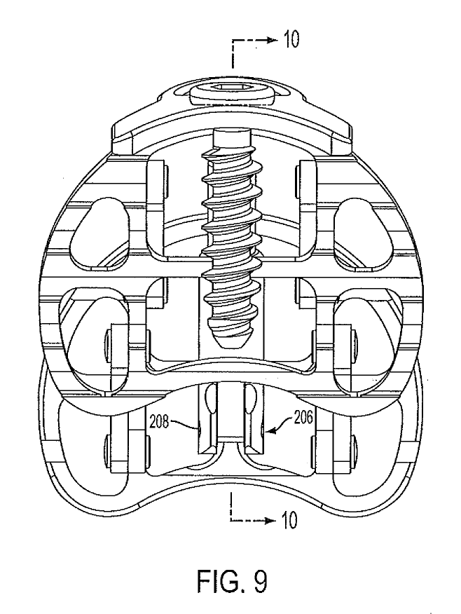

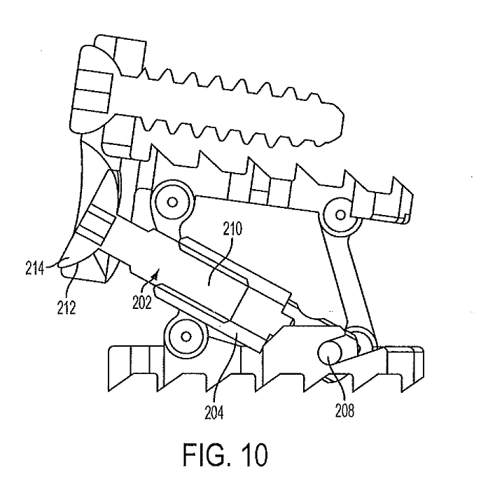

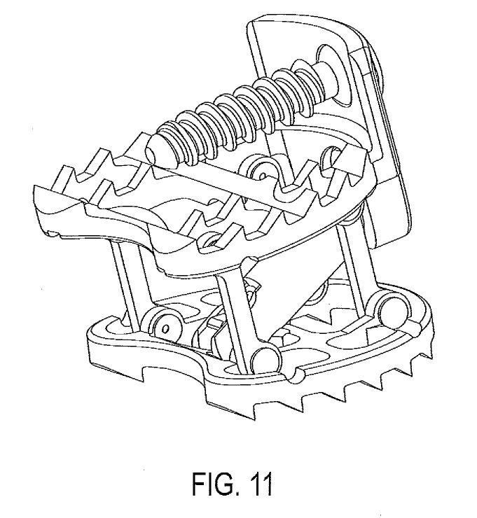

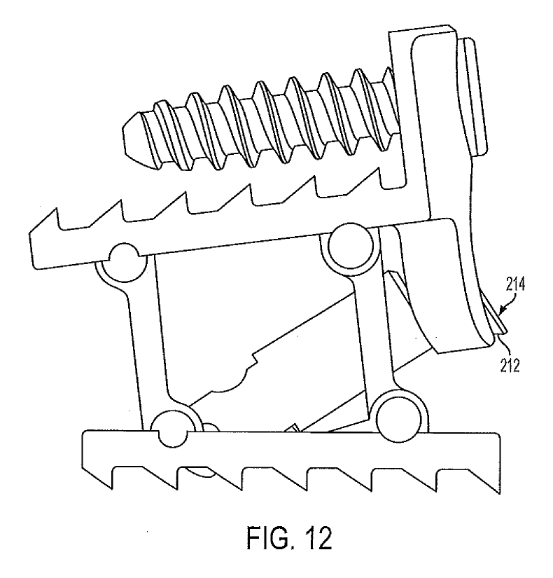

After the tool is disengaged, a lock 200 may be inserted to hold the implant extended. The exemplary lock further acts as an adjuster to permit a final adjustment of the degree of extension. The exemplary adjuster/lock comprises a screw mechanism The exemplary screw mechanism includes a screw 202 (FIG. 10) and an internally threaded sleeve or cylinder 204 receiving the screw. The end of the cylinder away from the screw head is provided with features for engaging the hook. In the exemplary embodiment, the end is formed as a clevis 206 (FIG. 9) carrying a pin 208 to capture the hook. Upon installation of the adjuster (e.g., in the relative extension of FIG. 10), the screw shaft/shank 210 may be screwed into the cylinder, with the underside 212 of the screw head 214 engaging the socket. Further tightening can further extend (FIGS. 11 and 12) the implant to a desired final extension. Thereafter, the implant can be filled (e.g., with prepared ground bone initially harvested during site preparation).

The components of the implant and tool may first be manufactured (e.g., via machining from appropriate metal stock) and then assembled. The exemplary implant materials are appropriate implantable materials such as titanium (including its alloys), cobalt (including its alloys), hastelloy, nitinol, and the like. More particular exemplary materials are medical grade titanium or titianium alloys.

One or more embodiments of the present invention have been described. Nevertheless, it will be understood that various modifications may be made without departing from the spirit and scope of the invention. For example, various features of existing or yet-developed implants may be incorporated. Additionally, features of the implant may be customized to the particular implant location or in view of the particular ailment being addressed. Accordingly, other embodiments are within the scope of the following claims.

What is claimed is:

1. A spinal implant, comprising:
a first bone-engaging member;
a second bone-engaging member; and
a linkage coupling the first bone-engaging member to the second bone-engaging member, wherein the implant includes means for receiving a tensile force to extend the implant.

2. The implant of claim 1 wherein:
a linkage is a four-bar linkage wherein the first and second bone-engaging members form respective first and second bars.

3. The implant of claim 1 wherein:
the means comprises a hook on one of the first and second members and a socket on the other of the first and second members.

4. The implant of claim 3 further comprising:
an adjuster having a first portion engaging the hook and a second portion engaging the socket.

5. A method for installing the implant of claim 1, the method comprising:
using a tensile adjustment tool engaging the means to extend the implant;
shimming the implant and removing the tool; and
installing a lock in place of the adjustment tool.

6. The method of claim 5 wherein the lock is an adjuster, the method further comprising: tightening the adjuster to further extend the implant.

7. A spinal implant comprising:
a first bone-engaging member;
a second bone-engaging member; and
a four-bar linkage coupling the first bone-engaging member to the second bone-engaging member.
